(19) United States
(12) Patent Application Publication
(10) Pub. No.: US 2021/0103859 A1
(43) Pub. Date: Apr. 8, 2021

(54) HEALTH CARE SYSTEM AND OPERATING METHOD THEREOF

(71) Applicant: Bonggeun SHIN, Gyeonggi-do (KR)

(72) Inventor: Bonggeun SHIN, Gyeonggi-do (KR)

(21) Appl. No.: 17/020,415

(22) Filed: Sep. 14, 2020

(30) Foreign Application Priority Data
Oct. 8, 2019 (KR) 10-2019-0124288

(51) Int. Cl. G06Q 10/02; G16H 80/00; G16H 40/67; G06N 20/00

(52) U.S. Cl. CPC G06Q 10/02; G06N 20/00; G16H 40/67; G16H 80/00

(57) ABSTRACT

Disclosed is a health care system that receives a treatment booth request from a user terminal of a user, transmits information on treatment booths located in the vicinity of a user to the user terminal, receives a treatment reservation request including information on a treatment booth selected by the user from the user terminal, makes a reservation for treatment of the user based on the received treatment reservation request, receives feedback information about the treatment from the user terminal, if the treatment of the user is completed in the selected treatment booth according to treatment information for the user, and transmits the received feedback information to a medical center terminal.

### CROSS-REFERENCE TO RELATED APPLICATION(S)

This application claims the benefit of Korean Patent Application No. 10-2019-0124288, filed on Oct. 8, 2019, in the Korean Intellectual Property Office, the disclosure of which is incorporated herein by reference.

### BACKGROUND

### 1. Field of the Invention

One or more example embodiments relate to a health care system.

### 2. Description of the Related Art

A patient visits a hospital to be treated by a doctor. However, if the hospital is located far away from the patient, it is difficult for the patient to visit the hospital. In addition, with the life extension and the advent of aging society, there is a shortage of specialists in rehabilitation and pain treatment. A new approach to rehabilitation and pain treatment is needed.

As a related art, there is Korean Patent Application Publication No. 10-2013-0051911 (Title: APPARATUS AND METHOD FOR PROVIDING HEALTH CARE SERVICE, Applicant: Samsung Electronics Co., Ltd.). In the publication, a method of providing a health care service, the method including calculating health data using sensor data from one or more sensors, processing the health data into an actuator control signal to be reflected in a corresponding actuator among one or more actuators according to a predetermined health service, and transmitting the actuator control signal to the corresponding actuator is disclosed.

### SUMMARY

According to an aspect, there is provided an operating method of a health care system, the operating method including receiving a treatment booth request from a user terminal of a user, transmitting information on treatment booths located in the vicinity of the user to the user terminal, and receiving a treatment reservation request including information on a treatment booth selected by the user from the user terminal, making a reservation for treatment of the user based on the received treatment reservation request, receiving feedback information about the treatment from the user terminal, if the treatment of the user is completed in the selected treatment booth according to treatment information for the user, and transmitting the received feedback information to a medical center terminal.

The operating method may further include receiving treatment completion information from the selected treatment booth, if the treatment is completed, and transmitting a feedback request about the treatment to the user terminal, if the treatment completion information is received.

The operating method may further include receiving second treatment information for the user from the medical center terminal, after transmitting the received feedback information to the medical center terminal.

The second treatment information may be the same as or different from the treatment information.

An artificial intelligence (AI) robot may be provided in the selected treatment booth.

The AI robot may be configured to treat the user in the selected treatment booth according to the received treatment information.

The treatment information may include information on a site of pain of the user, information on a treatment site of the user, and treatment method information.

The operating method may further include generating, if a reservation for the treatment is made, reservation information and transmitting the generated reservation information to the user terminal.

The operating method may further include generating, if a reservation for the treatment is made, temporary visitor code information based on identification (ID) information of the selected treatment booth, reservation date and time information for the treatment, and ID information of the user, mapping the ID information of the selected treatment booth, the reservation date and time information for the treatment, and the ID information of the user, recording the mapped information in a mapping table, and transmitting the generated temporary visitor code information to the user terminal, checking, if an admission verification request and information obtained by the selected treatment booth by scanning a visitor code of the user terminal are received from the selected treatment booth, whether the obtained information is present in the mapping table and whether the time of the admission verification request matches the reservation date and time information, and verifying an admission of the user based on a result of the checking.

According to an aspect, there is provided a health care system including a user terminal, and a health care server configured to receive a treatment booth request from the user terminal, transmit information on treatment booths located in the vicinity of a user to the user terminal, receive a treatment reservation request including information on a treatment booth selected by the user from the user terminal, make a reservation for treatment of the user based on the received treatment reservation request, receive feedback information about the treatment from the user terminal, if the treatment of the user is completed in the selected treatment booth according to treatment information for the user, and transmit the received feedback information to a medical center terminal.

The health care server may be configured to receive treatment completion information from the selected treatment booth, if the treatment is completed, and transmit a feedback request about the treatment to the user terminal, if the treatment completion information is received.

The health care server may be configured to receive second treatment information for the user from the medical center terminal, after transmitting the received feedback information to the medical center terminal.

The second treatment information may be the same as or different from the treatment information.

An AI robot may be provided in the selected treatment booth.

The AI robot may be configured to treat the user in the selected treatment booth according to the received treatment information.

The treatment information may include information on a site of pain of the user, information on a treatment site of the user, and treatment method information.

The health care server may be configured to generate, if a reservation for the treatment is made, reservation information and transmit the generated reservation information to the user terminal.

The health care server may be configured to generate, if a reservation for the treatment is made, temporary visitor code information based on ID information of the selected treatment booth, reservation date and time information for the treatment, and ID information of the user, map the ID information of the selected treatment booth, the reservation date and time information for the treatment, and the ID information of the user, record the mapped information in a mapping table, transmit the generated temporary visitor code information to the user terminal, check, if an admission verification request and information obtained by the selected treatment booth by scanning a visitor code of the user terminal are received from the selected treatment booth, whether the obtained information is present in the mapping table and whether the time of the admission verification request matches the reservation date and time information, and verify an admission of the user based on a result of the checking.

According to example embodiments, a robot may perform mild/moderate pain treatment, and specialized staff (for example, a doctor) may focus on patients having moderate or greater pain and rehabilitation patients, whereby the quality of treatment may increase.

According to example embodiments, pain treatment and management may be performed even in medically underserved areas.

According to example embodiments, a robot may perform treatment or procedures according to a determined protocol, whereby better results may be produced.

According to example embodiments, time of medical staff may be saved, and musculoskeletal pain and stress of the medical staff caused by repeated procedures may be reduced, whereby the overall quality of medical services may improve.

Additional aspects of example embodiments will be set forth in part in the description which follows and, in part, will be apparent from the description, or may be learned by practice of the disclosure.

### BRIEF DESCRIPTION OF THE DRAWINGS

These and/or other aspects, features, and advantages of the invention will become apparent and more readily appreciated from the following description of example embodiments, taken in conjunction with the accompanying drawings of which:

FIGS. 1 through 2B illustrate a health care system according to an example embodiment;

FIG. 3 illustrates an example of verifying an admission to a treatment booth according to an example embodiment;

FIGS. 4A and 4B illustrate examples of verifying an admission to a treatment booth according to an example embodiment;

FIG. 5 is a flow chart illustrating an operating method of a health care system according to an example embodiment;

FIG. 6 is a block diagram illustrating a health care server according to an example embodiment; and

FIGS. 7 to 11 illustrate a treatment booth according to an example embodiment.

### DETAILED DESCRIPTION

Hereinafter, example embodiments will be described in detail with reference to the accompanying drawings. However, various alterations and modifications may be made to the example embodiments. Here, the example embodiments are not construed as limited to the disclosure. The example embodiments should be understood to include all changes, equivalents, and replacements within the idea and the technical scope of the disclosure.

The terminology used herein is for the purpose of describing particular example embodiments only and is not to be limiting of the example embodiments. The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which example embodiments belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

When describing the example embodiments with reference to the accompanying drawings, like reference numerals refer to like elements and a repeated description related thereto will be omitted. In the description of example embodiments, detailed description of well-known related structures or functions will be omitted when it is deemed that such description will cause ambiguous interpretation of the present disclosure.

Also, in the description of the components, terms such as first, second, A, B, (a), (b) or the like may be used herein when describing components of the present disclosure. These terms are used only for the purpose of discriminating one element from another element, and the nature, the sequences, or the orders of the elements are not limited by the terms. When one element is described as being “connected”, “coupled”, or “attached” to another element, it should be understood that one element can be connected or attached directly to another element, and an intervening element can also be “connected”, “coupled”, or “attached” to the elements.

The same name may be used to describe an element included in the example embodiments described above and an element having a common function. Unless otherwise mentioned, the descriptions on the example embodiments may be applicable to the following example embodiments and thus, duplicated descriptions will be omitted for conciseness.

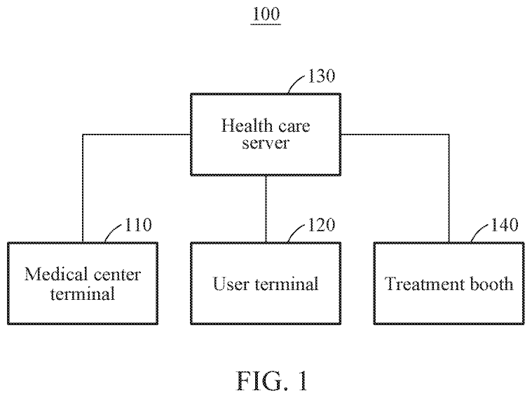

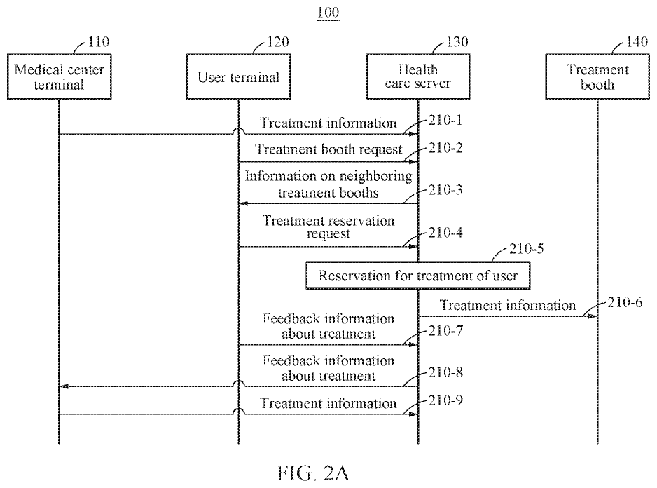

FIGS. 1 through 2B illustrate a health care system according to an example embodiment.

Referring to FIG. 1, a health care system 100 includes a medical center terminal 110, a user terminal 120, a health care server 130, and a treatment booth 140.

Although there are shown a single medical center terminal 110, a single user terminal 120, and a single treatment booth 140 in FIG. 1, the health care system 100 may include a plurality of medical center terminals, a plurality of user terminals, and a plurality of treatment booths.

A user may be diagnosed through telemedicine by a doctor or therapist (for example, physical therapist). That is, the doctor or physical therapist may diagnose the user through telemedicine.

The medical center terminal 110 is a terminal (for example, fixed terminal or mobile terminal) used by the doctor or therapist. The medical center terminal 110 may generate treatment information including a result of diagnosing the user by the doctor or physical therapist, a treatment site, and a treatment method, and transmit the generated treatment information to the health care server 130.

The user terminal 120 may be a terminal (for example, fixed terminal or mobile terminal) used by the user. The user terminal 120 may store a health care application for a service provided by the health care system 100.

The user may search for one or more neighboring treatment booths by utilizing the user terminal 120 (or the health care application) and reserve a treatment booth 140. In other words, the user terminal 120 may receive information on the treatment booths located in the vicinity of the user from the health care server 130, and request the health care server 130 to reserve a treatment booth. The health care server 130 may generate reservation information by reserving the treatment booth 140 among several treatment booths, and transmit the reservation information to the treatment booth 140.

The user may visit the treatment booth 140, perform a verification procedure (or visit procedure), and then be treated in the treatment booth 140. An artificial intelligence (AI) robot may be provided in the treatment booth 140. The AI robot may treat the user according to the treatment information received from the health care server 130.

The treatment booth 140 may operate in an unmanned manner, but is not limited thereto, and may operate in a manned manner.

After the treatment, the user may record a feedback on the treatment in the health care application. In other words, the user terminal 120 may transmit feedback information about the treatment to the health care server 130.

The health care server 130 may transmit the feedback information to the medical center terminal 110, and the doctor or physical therapist may check the feedback information and retain or change the treatment information.

Hereinafter, examples of the operation of the health care system 100 will be described with reference to FIGS. 2A and 2B.

FIG. 2A shows an example of the operation of the health care system 100.

Referring to FIG. 2A, in operation 210-1, the medical center terminal 110 transmits treatment information to the health care server 130. The treatment information may include, for example, one or more of a result of diagnosing a user by a doctor or therapist through telemedicine, a treatment method, a treatment site of the user, a site of pain of the user, and precautions for treatment.

In operation 210-2, the user terminal 120 transmits a treatment booth request to the health care server 130. For example, the user may run a health care application after the telemedicine and select a treatment booth item in the health care application, and based on the selection, the user terminal 120 may request the health care server 130 to search treatment booths (or to provide information on neighboring treatment booths).

In operation 210-3, the health care server 130 transmits the information on the neighboring treatment booths to the user terminal 120. For example, when the treatment booth request is received from the user terminal 120, the health care server 130 may search for one or more treatment booths located in the vicinity (for example, within a radius of 10 km) of the user, and transmit information (for example, available dates and times, locations, directions, and the like) related to the found one or more treatment booths to the user terminal 120. The user terminal 120 may display the information on the found one or more treatment booths on a display.

In operation 210-4, the user terminal 120 transmits, to the health care server 130, a treatment reservation request including information on a treatment booth 140 selected by the user from among the found one or more treatment booths. For example, the user may transmit, to the health care server 130, selection information indicating that the user selects the treatment booth 140 closest to the user from among the found one or more treatment booths. In addition, the treatment reservation request may include reservation date and time information for treatment. For example, if the user inputs 2 p.m. a week from the current time, as treatment reservation date and time, into the user terminal 120, the user terminal 120 may transmit the treatment reservation date and time information (that is, 2 p.m. a week from the current time) to the health care server 130.

In operation 210-5, the health care server 130 makes a reservation for treatment of the user based on the treatment reservation request. For example, if the treatment reservation location in the treatment reservation request is set as the treatment booth 140, and the treatment reservation date and time in the treatment reservation request is set as 2 p.m. a week from the current time, the health care server 130 may reserve the treatment booth 140 such that the user may be treated at 2 p.m. a week from the current time.

If the reservation is made for treatment of the user, the health care server 130 may generate reservation information corresponding to the treatment reservation request and transmit the generated reservation information to the user terminal 120. The user may confirm that the reservation for treatment has been made exactly.

In operation 210-6, the health care server 130 transmits the treatment information received from the medical center terminal 110 to the reserved treatment booth 140. In addition, the health care server 130 transmits reservation information corresponding to the treatment reservation request to the treatment booth 140.

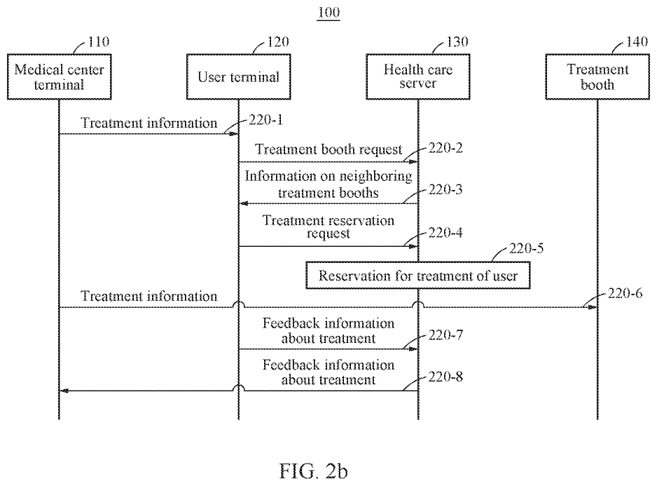

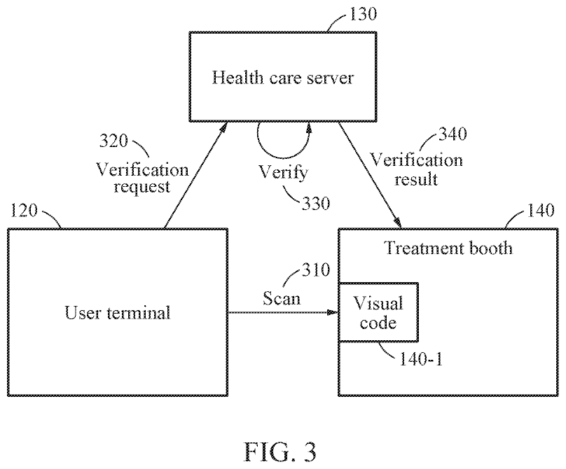

The user may visit the treatment booth 140 at the reservation date and time, perform a predetermined verification procedure (or visit procedure), and then enter the treatment booth 140. For example, the user may enter the treatment booth 140 if an admission verification (for example, password-based user verification, or biometric information-based user verification) is successful. In another example, the user may enter the treatment booth 140 through admission verification, which will be described with reference to FIG. 3 or FIGS. 4A and 4B.

An AI robot in the treatment booth 140 treats the user according to the treatment information received from the health care server 130. For example, a pain treatment laser may be provided in the treatment booth 140, and the AI robot may treat the site of pain (or treatment site) of the user with the pain treatment laser.

After the treatment is completed, the user may pay for the treatment at the treatment booth 140. Depending on the implementation, the payment may be performed through the health care application.

After the treatment is completed, the user may record a feedback on the treatment in the health care application. For example, the user may record, in the health care application, a feedback that the pain has decreased through the treatment, a feedback that the user still has pain, or a feedback that the user has pain in another part. In operation 210-7, the user terminal 120 transmits feedback information about the treatment to the health care server 130.

After the treatment is completed, the health care server 130 may receive treatment completion information from the treatment booth 140, and when the treatment completion information is received, request the user terminal 120 for a feedback on the treatment.

In operation 210-8, the health care server 130 transmits the feedback information about the treatment to the medical center terminal 110.

Depending on the implementation, the user terminal 120 may transmit the feedback information about the treatment to the medical center terminal 110.

The doctor or therapist may determine whether to change a treatment method by checking the feedback information about the treatment. In this example, the doctor or therapist may receive a more specific feedback on the treatment from the user through telemedicine, and determine whether to change the treatment method for the user. The doctor or therapist may retain or change the treatment information through the medical center terminal 110.

In operation 210-9, the medical center terminal 110 transmits the treatment information for the user to the health care server 130. If the doctor or therapist does not change the treatment method, the treatment information in operation 210-9 is the same as the treatment information in operation 210-1, and if the doctor or therapist changes the treatment method, the treatment information in operation 210-9 is different from the treatment information in operation 210-1. Depending on the implementation, if the doctor or therapist determines that there is no longer a need for treatment, the medical center terminal 110 may not transmit treatment information to the health care server 130.

After operation 210-9, operations 210-2 to 210-8 may be performed.

FIG. 2B shows another example of the operation of the health care system 100.

In operation 220-1, the medical center terminal 110 transmits treatment information to the user terminal 120. The treatment information may include, for example, one or more of a result of diagnosing a user by a doctor or therapist through telemedicine, a treatment method, a treatment site of the user, a site of pain of the user, and precautions for treatment.

In operation 220-2, the user terminal 120 transmits a treatment booth request to the health care server 130. The description of operation 210-2 may apply to operation 220-2, and thus a detailed description of operation 220-2 will be omitted.

In operation 220-3, the health care server 130 transmits the information on the neighboring treatment booths to the user terminal 120. The description of operation 210-3 may apply to operation 220-3, and thus a detailed description of operation 220-3 will be omitted.

In operation 220-4, the user terminal 120 transmits, to the health care server 130, a treatment reservation request including information on a treatment booth 140 selected by the user from among the found one or more treatment booths. The description of operation 210-4 may apply to operation 220-4, and thus a detailed description of operation 220-4 will be omitted.

In operation 220-5, the health care server 130 makes a reservation for treatment of the user based on the treatment reservation request. The description of operation 210-5 may apply to operation 220-5, and thus a detailed description of operation 220-5 will be omitted.

If the reservation is made for treatment of the user, the health care server 130 may generate reservation information corresponding to the treatment reservation request and transmit the generated reservation information to the medical center terminal 110.

When the reservation information is received from the health care server 130, the medical center terminal 110 may transmit the treatment information to the treatment booth 140, in operation 220-6. Depending on the implementation, the medical center terminal 110 may transmit the treatment information to the treatment booth 140 through the health care server 130.

The user may visit the treatment booth 140 at the reservation date and time, perform a predetermined verification procedure (or visit procedure), and then enter the treatment booth 140.

After the treatment is completed in the treatment booth 140, the user may record a feedback on the treatment in the health care application, and the user terminal 120 transmits feedback information about the treatment to the health care server 130, in operation 220-7. The description of operation 210-7 may apply to operation 220-7, and thus a detailed description of operation 220-7 will be omitted.

In operation 220-8, the health care server 130 transmits the feedback information about the treatment to the medical center terminal 110.

FIG. 3 illustrates an example of verifying an admission to a treatment booth according to an example embodiment.

Referring to FIG. 3, a visual code 140-1 (for example, a quick response (QR) code) is attached to a treatment booth 140. Identification (ID) information of the treatment booth 140 may be encoded in the visual code 140-1.

When the user visits the treatment booth 140 at reservation date and time, the user may bring the user terminal 120 close to the visual code 140-1 such that a camera of the user terminal 120 may scan the visual code 140-1.

In operation 310, the user terminal 120 may scan the visual code 140-1 to obtain or extract ID information from the visual code 140-1.

In operation 320, the user terminal 120 may transmit the obtained ID information to the health care server 130 to request the health care server 130 to verify an admission.

In operation 330, the health care server 130 may verify an admission of the user based on the ID information received from the user terminal 120. In operation 340, the health care server 130 may transmit a verification result to the treatment booth 140. For example, the health care server 120 may determine whether date and time information (hereinafter, the first date and time information) when the ID information is received from the user terminal 120 matches date and time information in the reservation information generated in operation 210-5, and whether the ID information received from the user terminal matches the ID information of the treatment booth 140 in the reservation information. Here, if the first date and time information matches the date and time information in the reservation information and the received ID information matches the ID information of the treatment booth 140 in the reservation information, the health care server 120 may verify that the user may enter or exit the treatment booth 140. In addition, the health care server 120 may transmit, to the treatment booth 140, a verification result indicating that the user may enter or exit the treatment booth 140. The treatment booth 140 may open a door such that the user may enter the treatment booth 140 and be treated by an AI robot. If at least one of the ID information and the first date and time information received from the user terminal 120 does not match the reservation information, the health care server 120 may disallow an admission of the user and transmit a verification failure to the treatment booth 140. In this example, the treatment booth 140 does not open the door. The treatment booth 140 may inform the user that the door cannot be opened due to the mismatching to the reservation information.

FIG. 4A illustrates an example of verifying an admission to a treatment booth according to an example embodiment.

Referring to FIG. 4A, the healthcare server 130 may generate temporary visitor code information based on at least one of ID information of the treatment booth 140, reservation date and time information for treatment of a user, and ID information of the user. For example, when a reservation is made for treatment through operation 210-5 of FIG. 2A, the healthcare server 130 may generate temporary visitor code information by combining ID information ax01 of the treatment booth 140, reservation date and time information 2019txy for treatment of the user, and ID information abc of the user. Then, the health care server 130 may map the ID information ax01 of the treatment booth 140, the reservation date and time information 2019txy for treatment of the user, and the ID information abc of the user to each other, and record the mapped information in a mapping table.

In operation 410-1, the health care server 130 may transmit the generated temporary visitor code information to the user terminal 120.

The user terminal 120 may display a visitor code 120-1 on the display based on the temporary visitor code information.

In operation 410-2, the camera of the treatment booth 140 may scan the visitor code 120-1 disposed on the display of the user terminal 120. In operation 410-3, the treatment booth 140 may request the health care server 130 to verify an admission of the user based on information obtained by scanning the visitor code 120-1. The information obtained by scanning the visitor code 120-1 may include the ID information of the treatment booth 140, the reservation date and time information for treatment of the user, and the ID information of the user.

The health care server 130 may verify an admission based on the mapping table and the information obtained by the treatment booth 140 by scanning the visitor code 120-1. For example, if the information obtained by the treatment booth 140 by scanning the visitor code 120-1 includes the ID information ax01 of the treatment booth 140, the reservation date and time information 2019txy for treatment of the user, and the ID information abc of the user, the health care server 130 may check whether ax01, 2019txy, and abc are mapped to each other in the mapping table, and check whether the reservation date and time information 2019txy matches the verification request time. If ax01, 2019txy, and abc are mapped to each other in the mapping table and the reservation date and time information 2019txy matches the verification request time, the health care server 130 may allow an admission of the user. If the reservation date and time information 2019txy does not match the verification request time, the health care server 130 may disallow an admission of the user.

In operation 410-4, the health care server 130 may transmit a verification result to the treatment booth 140.

The treatment booth 140 opens a door when the verification result indicates that the admission is allowed. The user may enter the treatment booth 140 and be treated by the AI robot. The treatment booth 140 does not open the door when the verification result indicates that the admission is disallowed. In this example, the treatment booth 140 may inform the user that the door cannot be opened because a reservation of the user is not found.

Referring to FIG. 4B, the user may visit the treatment booth 140, and then run a health care application and select an admission verification item in the health care application. In this example, the user terminal 120 may display a verification code 120-2 on the display. The treatment information as described above may be stored in the user terminal 120.

The verification code 120-2 may be randomly generated by the health care application, or may be received from the health care server 130. The verification code 120-2 may be, for example, a QR code, or a random n-digit code.

The treatment booth 140 may scan the verification code 120-2. Depending on the implementation, the user may input the n-digit code into the treatment booth 140.

In operation 420-1, the treatment booth 140 may transmit the information obtained by scanning the verification code 120-2 (or the input n-digit code) to the health care server 130 and request the health care server 130 to verify an admission.

The health care server 130 may verify an admission of the user based on the information obtained by scanning the verification code 120-2 (or the n-digit code input into the treatment booth 140).

In operation 420-2, the health care server 130 may transmit a verification result to the treatment booth 140. Depending on the implementation, the health care server 130 may transmit the treatment information of the user along with the verification result to the treatment booth 140. In other words, unlike the examples described above with reference to FIGS. 2A and 2B, the health care server 130 may transmit the treatment information of the user to the treatment booth 140 when the admission of the user is verified successfully.

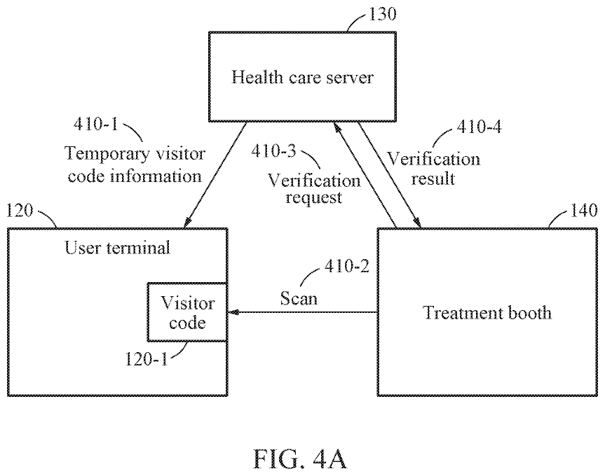

FIG. 5 is a flow chart illustrating an operating method of a health care system according to an example embodiment.

Referring to FIG. 5, in operation 510, the health care server 130 receives treatment information for a user from the medical center terminal 110.

In operation 520, the health care server 130 receives a treatment booth request from the user terminal 120 of the user.

In operation 530, the health care server 130 transmits information on treatment booths located in the vicinity of the user to the user terminal 120, and receives a treatment reservation request including information on a treatment booth 140 selected by the user from the user terminal 120.

In operation 540, the health care server 130 makes a reservation for treatment of the user based on the received treatment reservation request.

In operation 550, the health care server 130 transmits the received treatment information to the selected treatment booth 140.

When the treatment for the user is completed in the selected treatment booth 140 according to the received treatment information, the health care server 130 receives feedback information about the treatment from the user terminal 120, in operation 560.

In operation 570, the health care server 130 transmits the received feedback information to the medical center terminal 110.

After transmitting the received feedback information to the medical center terminal 110, the health care server 130 may receive second treatment information for the user from the medical center terminal 110. In this example, the second treatment information may be the same as or different from the treatment information.

When the treatment of the user is completed in the selected treatment booth 140, the health care server 130 may receive treatment completion information from the selected treatment booth 140, and when receiving the treatment completion information, transmit a feedback request about the treatment to the user terminal 120.

An AI robot may be provided in the selected treatment booth 140. Here, the AI robot may treat the user according to the treatment information received by the selected treatment booth 140.

The treatment information may include information on a site of pain of the user, information on a treatment site of the user, and treatment method information.

If a reservation for treatment is made, the health care server 130 may generate reservation information and transmit the generated reservation information to the user terminal 120.

If a reservation for treatment is made, the health care server 130 may generate temporary visitor code information based on ID information of the selected treatment booth 140, reservation date and time information for the treatment, and ID information of the user. The health care server 130 may map the ID information of the selected treatment booth 140, the reservation date and time information for the treatment, and the ID information of the user, record the mapped information in a mapping table, and transmit the generated temporary visitor code information to the user terminal 120. When information obtained by the selected treatment booth 140 by scanning a visitor code of the user terminal 120 and an admission verification request are received from the selected treatment booth 140, the health care server 130 may check whether the obtained information is present in the mapping table and whether the time of the admission verification request matches the reservation date and time information, and verify an admission of the user based on a result of the checking.

The description provided with reference to FIGS. 1 through 4B also applies to the description of FIG. 5, and thus a detailed description will be omitted for conciseness.

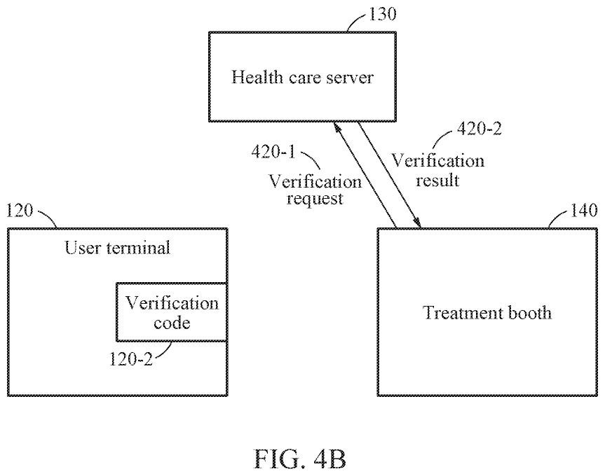

FIG. 6 is a block diagram illustrating a health care server according to an example embodiment.

Referring to FIG. 6, the health care server 130 includes a memory 610 and a controller 620.

The controller 620 receives treatment information for a user from the medical center terminal 110 through a communication interface.

The controller 620 receives a treatment booth request from the user terminal 120 of the user through the communication interface.

The controller 620 transmits information on treatment booths located in the vicinity of the user to the user terminal 120 through the communication interface.

The controller 620 receives a treatment reservation request including information on a treatment booth 140 selected by the user from the user terminal 120 through the communication interface, and makes a reservation for treatment of the user based on the received treatment reservation request.

The controller 620 transmits the received treatment information to the selected treatment booth 140 through the communication interface, and when the treatment for the user is completed in the selected treatment booth 140 according to the received treatment information, receives feedback information about the treatment from the user terminal 120 through the communication interface.

The controller 620 transmits the received feedback information to the medical center terminal 110 through the communication interface.

The description provided with reference to FIGS. 1 through 5 also applies to the description of FIG. 6, and thus a detailed description will be omitted for conciseness.

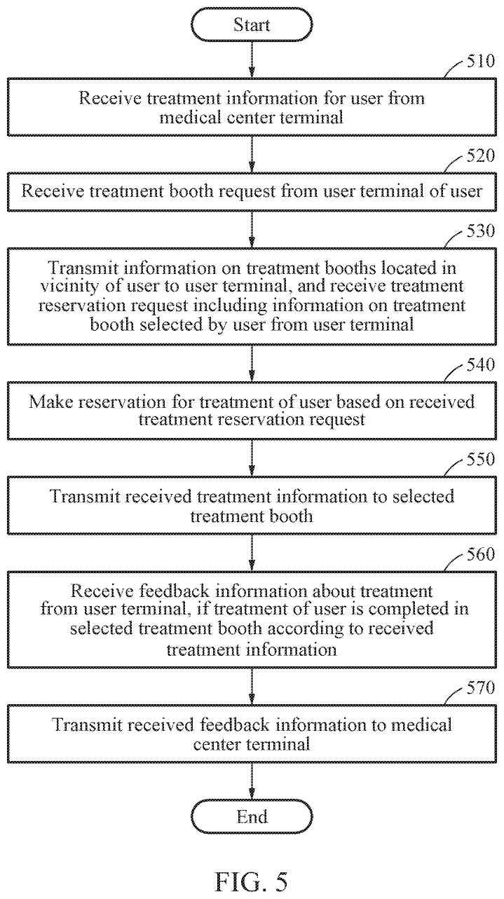

FIGS. 7 to 11 illustrate a treatment booth according to an example embodiment.

Referring to FIG. 7, the treatment booth 140 includes an AI robot 710 and a treatment device 720.

The AI robot 710 and the treatment device 720 may be physically separate from each other, or in an integral form.

The treatment device 720 may be a device for pain treatment, cosmetic treatment, or obesity treatment. Depending on the implementation, two or more of a treatment device for pain treatment, a treatment device for cosmetic treatment, and a treatment device for obesity treatment may be provided in the treatment booth 140.

The treatment device 720 may output laser beams, ultrasonic waves, high-frequency waves, low-frequency waves, or light for treatment. The treatment device 720 may output laser beams, ultrasonic waves, high-frequency waves, low-frequency waves, or light to a site to be treated under the control of the AI robot 710.

The AI robot 710 may check the condition of the treated user, and transmit the condition of the treated user to the medical center terminal 110. For example, the AI robot 710 may record a treatment activity and transmit an image generated by the recording to the medical center terminal 110. The user may take an image of the treated site and upload the image through a health care application. The image may be transmitted to the medical center terminal 110.

A doctor or therapist may give an appropriate prescription according to the condition of the treated user, and the user may then visit the treatment booth 140 and be treated by the AI robot 710 according to the prescription.

The AI robot 710 may perform the operation of the treatment booth 140 described through FIGS. 1 to 6, in addition to the treatment.

Depending on the implementation, the treatment booth 140 may further include a management device. The management device may perform the operation of the treatment booth 140 described with reference to FIGS. 1 to 6. For example, the management device may receive treatment information and reservation information from the health care server 130, and open a door of the treatment booth 140 when the admission of the user is verified.

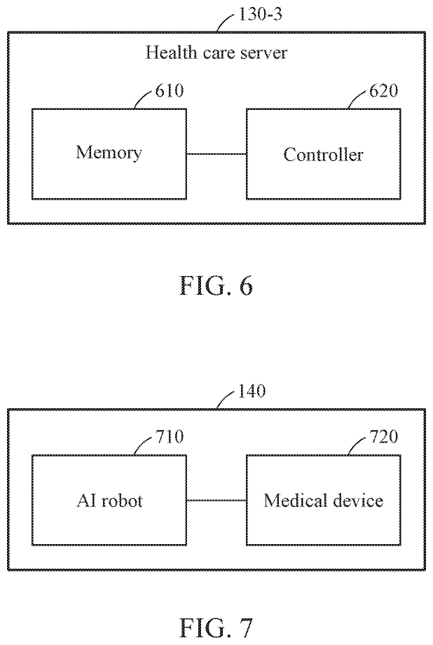

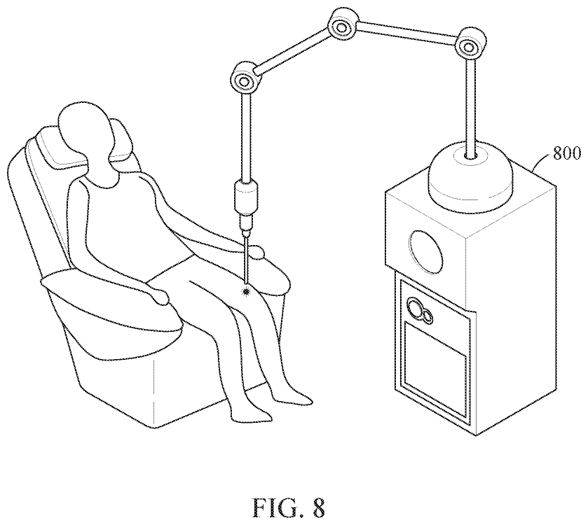

The AI robot 710 and the treatment device 720 may be integrated into equipment 800 shown in FIGS. 8 and 9. The equipment 800 may move by itself. Depending on the implementation, the management device described through FIG. 7 may be included in the equipment 800.

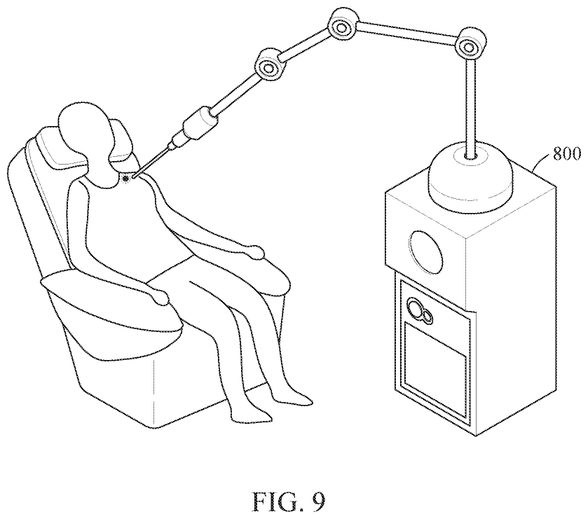

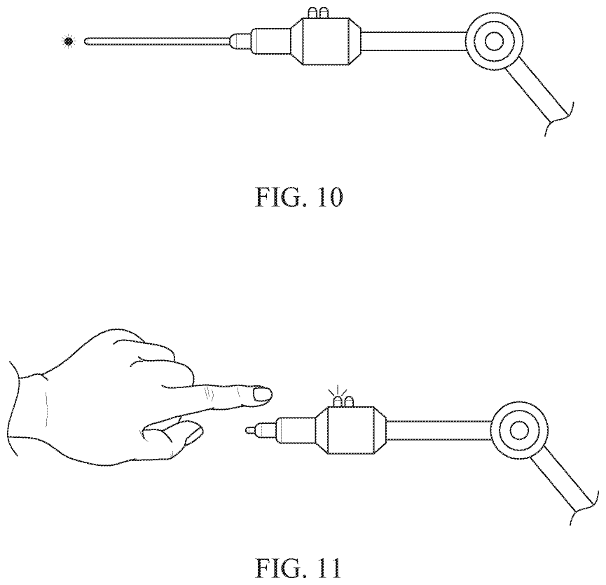

The treatment device 720 of the equipment 800 may output a laser beam as shown in FIG. 10. The treatment device 720 of the equipment 800 may include a detection sensor, and when the detection sensor detects that the user approaches the laser beam while the laser beam is being output, the treatment device 720 may stop outputting the laser beam as shown in FIG. 11.

The AI robot 710 may identify characteristics (for example, skin color and/or the presence or absence of hair) of the user, and change a portion of the treatment method included in the treatment information based on the identified characteristics. Accordingly, the AI robot 710 may perform treatment accurately according to the characteristics of the user.

The AI robot 710 may identify the characteristics of the user and perform optimal treatment on a treatment site of the user. For example, in the case of a user having tennis elbow, the AI robot 710 may treat the user considering gender, height, elbow bone shape, elbow bone flexion, and elbow bone size of the user.

The AI robot 710 may treat the user using the treatment device 720. In this example, the AI robot 710 may sense a tension of the user, or sense the user moving to avoid the laser beam output from the treatment device 720. Based on a result of the sensing, the AI robot 710 may temporarily stop using the treatment device 720.

When the user moves while being treated, the AI robot 710 may temporarily stop the operation of the treatment device 720 and restart the treatment when the user comes to the right position.

The AI robot 710 may accurately identify and sense the treatment site prescribed by the doctor and radiate a laser beam to the treatment site.

In an example, a treatment chair that the user may sit on when being treated (or a treatment bed that the user may lie down on when being treated) may be provided in the treatment booth 140. The AI robot 710 may adjust the tilt of the backrest of the treatment chair or the height of the treatment chair based on the treatment information of the user such that a laser beam may be radiated to the treatment site of the user. For example, when the treatment site of the user is a specific part of the thigh, the AI robot 710 may guide the user to sit on the treatment chair, and when the user sits on the treatment chair, adjust the tilt of the backrest of the treatment chair such that a laser beam may be radiated to the site to be treated.

An example of a user visiting the treatment booth 140 for pain treatment will be described.

When the user visits the treatment booth 140, the AI robot 710 may guide the user to a treatment room. There may be a treatment bed and/or a treatment chair in the treatment room.

The AI robot 710 may identify a site of pain of the user based on treatment information of the user. In other words, the AI robot 710 may identify a treatment site of the user based on the treatment information.

The AI robot 710 may determine a treatment pose of the user based on the treatment information (or diagnostic information) of the user, and may request the user to take the determined treatment pose. For example, if the treatment site of the user is the knee, the AI robot 710 may determine the treatment pose of the user to be “lying on the treatment bed and exposing the knee”, and request the user to take the determined treatment pose. In other words, the AI robot 710 may guide the user to lie down on the treatment bed and expose the knee. In another example, if the treatment site of the user is the lower back, the AI robot 710 may determine the treatment pose of the user to be “lying down (or lying on the side) on the treatment bed and exposing the lower back”, and request the user to take the determined treatment pose. In other words, the AI robot 710 may guide the user to lie down or lie on the side on the treatment bed and expose the lower back.

The AI robot 710 may check the pose of the user through a camera, and scan the treatment site when the checked pose matches the determined treatment pose. If the checked pose does not match the determined treatment pose, the AI robot 710 may request the user to move to take the determined pose.

The AI robot 710 may scan the treatment site of the user. For example, the AI robot 710 may scan the treatment site of the user through the camera and/or the sensor. The AI robot 710 may identify the skin characteristics (for example, skin color and skin tone) of the user through scanning. In addition, the AI robot 710 may accurately identify or determine an area (or treatment area) to which a laser beam is to be radiated based on a result of the scanning.

The AI robot 710 may control the treatment bed or the treatment chair so that the laser beam may be accurately radiated to the area to which the laser beam is to be radiated. For example, if the user lies down on the treatment bed and exposes the knee, the AI robot 710 may accurately identify or determine the area to which the laser beam is to be radiated by scanning the knee, and control the treatment bed such that the laser beam may be accurately radiated to the identified area.

The AI robot 710 may determine an intensity of the treatment based on the result of the scanning. In response to the determination that the skin color of the user is dark through scanning, the AI robot 710 may determine an intensity of the laser beam by referring to a table. Here, the intensity of the laser beam and the skin color are mapped in the table.

The AI robot 710 may monitor the condition of the user while the user is being treated, and temporarily stop the treatment based on a result of the monitoring. For example, if the user frowns, or a movement of the user results in the treatment site being out of the area to which the laser beam is radiated, the AI robot 710 may determine that the user feels pain, and temporarily stop the treatment. In other words, the AI robot 710 may detect whether the user feels pain due to the treatment based on the result of the monitoring, and in response to the detection that the user feels pain due to the treatment, temporarily stop the treatment.

If the user moves while being treated, the AI robot 710 may temporarily stop the treatment, and control the treatment bed or the treatment chair or relocate the treatment device 720 so that the laser beam may be accurately radiated to the treatment site of the user.

The AI robot 710 may converse with the user. For example, the AI robot 710 may ask the user if the user feels pain, or inform the user of the time remaining until the treatment is completed. If the user says it hurts due to the treatment, the AI robot 710 may temporarily stop the treatment.

When the treatment is over, the AI robot 710 may inform the user about the care needed after the treatment. Information about the care needed after the treatment may be transmitted to the user terminal.

An example of a user visiting the treatment booth 140 for cosmetic treatment will be described.

The cosmetic treatment includes skin treatment, hair removal treatment, and body beauty treatment, but is not limited thereto.

When the user visits the treatment booth 140, the AI robot 710 may guide the user to a treatment room. There may be a treatment bed and/or a treatment chair in the treatment room.

The AI robot 710 may identify a site of the user on which cosmetic treatment is to be performed based on treatment information (or diagnostic information) of the user. In other words, the AI robot 710 may identify a cosmetic treatment site of the user based on the treatment information.

The AI robot 710 may determine a treatment pose of the user based on the treatment information (or diagnostic information) of the user, and may request the user to take the determined treatment pose. For example, if the cosmetic treatment site of the user is the skin under the eyes, the AI robot 710 may determine the treatment pose of the user to be “lying on the treatment bed”, and request the user to take the determined treatment pose. In other words, the AI robot 710 may guide the user to lie down on the treatment bed.

The AI robot 710 may check the pose of the user through a camera, and scan the treatment site when the checked pose matches the determined treatment pose. If the checked pose does not match the determined treatment pose, the AI robot 710 may request the user to move to take the determined pose.

The AI robot 710 may scan the cosmetic treatment site of the user. For example, the AI robot 710 may scan the cosmetic treatment site of the user through the camera and/or the sensor. The AI robot 710 may identify the characteristics (for example, at least one of skin tone, skin curve, face size, the presence or absence of hair, contour of the face, and body shape) of the user through scanning. In addition, the AI robot 710 may accurately identify an area (or treatment area) to which a laser beam is to be radiated based on a result of the scanning.

The AI robot 710 may control the treatment bed or the treatment chair so that the laser beam may be accurately radiated to the cosmetic treatment site of the user. For example, if the user lies down on the treatment bed, the AI robot 710 may accurately identify the area to which the laser beam is to be radiated through scanning, and control the treatment bed such that the laser beam may be accurately radiated to the identified area.

The AI robot 710 may determine an intensity of the treatment based on the result of the scanning. In response to the determination that the skin color of the user is dark through scanning, the AI robot 710 may determine an intensity of the laser beam by referring to a table. Here, the intensity of the laser beam and the skin color are mapped in the table.

The AI robot 710 may apply a cooling gel to the cosmetic treatment site of the user (or the area to which the laser beam is to be radiated) or to a wider area than the cosmetic treatment site of the user (or the area to which the laser beam is to be radiated).

The AI robot 710 may radiate a laser beam to the cosmetic treatment site (or the area to which the laser beam is to be radiated).

The AI robot 710 may monitor the condition of the user while the user is being cosmetically treated, and temporarily stop the treatment based on a result of the monitoring. For example, if the user frowns, or a movement of the user results in the treatment site being out of the area to which the laser beam is radiated, the AI robot 710 may determine that the user feels pain, and temporarily stop the treatment. In other words, the AI robot 710 may detect whether the user feels pain due to the treatment based on the result of the monitoring, and in response to the detection that the user feels pain due to the treatment, temporarily stop the treatment. The AI robot 710 may perform preventive measure during the treatment using a nerve detector. For example, the AI robot 710 may measure brainwaves of the user using the nerve detector, and perform preventive measure (for example, temporarily stopping the treatment, or reducing the intensity of the laser beam) in response to a change in the brainwaves.

If the user moves while being treated, the AI robot 710 may temporarily stop the treatment, and control the treatment bed or the treatment chair or relocate the treatment device so that the laser beam may be accurately radiated to the treatment site of the user.

The AI robot 710 may converse with the user. For example, the AI robot 710 may ask the user if the user feels pain, or inform the user of the time remaining until the treatment is completed. If the user says it hurts due to the treatment, the AI robot 710 may temporarily stop the treatment.

If smoke is generated by the cosmetic treatment, the AI robot 710 may operate a smoke evacuator.

When the treatment is over, the AI robot 710 may cool the treated site. For example, the AI robot 710 may place a pack on the treated site.

When the treatment is over, the AI robot 710 may inform the user about the care needed after the treatment. Information about the care needed after the treatment may be transmitted to the user terminal.

The description of pain treatment and cosmetic treatment may also apply to obesity treatment.

In addition to the laser beams, ultrasonic waves, high-frequency waves, low-frequency waves, or light may be used for pain treatment and cosmetic treatment.

Treatment using the AI robot 710 may be performed independently in the treatment booth 140, or may be performed in conjunction with medical staff at a hospital.

The description provided with reference to FIGS. 1 through 6 may also apply to the description of FIGS. 7 to 11, and thus a detailed description will be omitted for conciseness.

The methods according to the above-described example embodiments may be recorded in non-transitory computer-readable media including program instructions to implement various operations of the above-described example embodiments. The media may also include, alone or in combination with the program instructions, data files, data structures, and the like. The program instructions recorded on the media may be those specially designed and constructed for the purposes of example embodiments, or they may be of the kind well-known and available to those having skill in the computer software arts. Examples of non-transitory computer-readable media include magnetic media such as hard disks, floppy disks, and magnetic tape; optical media such as CD-ROM discs, DVDs, and/or Blue-ray discs; magneto-optical media such as optical discs; and hardware devices that are specially configured to store and perform program instructions, such as read-only memory (ROM), random access memory (RAM), flash memory (e.g., USB flash drives, memory cards, memory sticks, etc.), and the like. Examples of program instructions include both machine code, such as produced by a compiler, and files containing higher-level code that may be executed by the computer using an interpreter. The above-described devices may be configured to act as one or more software modules in order to perform the operations of the above-described example embodiments, or vice versa.

The software may include a computer program, a piece of code, an instruction, or some combination thereof, to independently or uniformly instruct or configure the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network-coupled computer systems so that the software is stored and executed in a distributed fashion. The software and data may be stored by one or more non-transitory computer-readable recording mediums.

A number of example embodiments have been described above. Nevertheless, it should be understood that various modifications may be made to these example embodiments. For example, suitable results may be achieved if the described techniques are performed in a different order and/or if components in a described system, architecture, device, or circuit are combined in a different manner and/or replaced or supplemented by other components or their equivalents.

Accordingly, other implementations are within the scope of the following claims.

What is claimed is:

1. An operating method of a health care system, the operating method comprising:
receiving a treatment booth request from a user terminal of a user;
transmitting information on treatment booths located in the vicinity of the user to the user terminal, and receiving a treatment reservation request including information on a treatment booth selected by the user from the user terminal;
making a reservation for treatment of the user based on the received treatment reservation request;
receiving feedback information about the treatment from the user terminal, if the treatment of the user is completed in the selected treatment booth according to treatment information for the user; and
transmitting the received feedback information to a medical center terminal.

2. The operating method of claim 1, further comprising:
receiving treatment completion information from the selected treatment booth, if the treatment is completed; and
transmitting a feedback request about the treatment to the user terminal, if the treatment completion information is received.

3. The operating method of claim 1, further comprising:
receiving second treatment information for the user from the medical center terminal, after transmitting the received feedback information to the medical center terminal,
wherein the second treatment information is the same as or different from the treatment information.

4. The operating method of claim 1, wherein an artificial intelligence (AI) robot is provided in the selected treatment booth, and
the AI robot is configured to treat the user in the selected treatment booth according to the received treatment information.

5. The operating method of claim 1, wherein the treatment information includes information on a site of pain of the user, information on a treatment site of the user, and treatment method information.

6. The operating method of claim 1, further comprising:
generating, if a reservation for the treatment is made, reservation information and transmitting the generated reservation information to the user terminal.

7. The operating method of claim 1, further comprising:
generating, if a reservation for the treatment is made, temporary visitor code information based on identification (ID) information of the selected treatment booth, reservation date and time information for the treatment, and ID information of the user;
mapping the ID information of the selected treatment booth, the reservation date and time information for the treatment, and the ID information of the user, recording the mapped information in a mapping table, and transmitting the generated temporary visitor code information to the user terminal;
checking, if an admission verification request and information obtained by the selected treatment booth by scanning a visitor code of the user terminal are received from the selected treatment booth, whether the obtained information is present in the mapping table and whether the time of the admission verification request matches the reservation date and time information; and
verifying an admission of the user based on a result of the checking.

8. A health care system, comprising:
a user terminal; and
a health care server configured to receive a treatment booth request from the user terminal, transmit information on treatment booths located in the vicinity of a user to the user terminal, receive a treatment reservation request including information on a treatment booth selected by the user from the user terminal, make a reservation for treatment of the user based on the received treatment reservation request, receive feedback information about the treatment from the user terminal, if the treatment of the user is completed in the selected treatment booth according to treatment information for the user, and transmit the received feedback information to a medical center terminal.

9. The health care system of claim 8, wherein the health care server is configured to receive treatment completion information from the selected treatment booth, if the treatment is completed, and transmit a feedback request about the treatment to the user terminal, if the treatment completion information is received.

10. The health care system of claim 8, wherein the health care server is configured to receive second treatment information for the user from the medical center terminal, after transmitting the received feedback information to the medical center terminal, and
the second treatment information is the same as or different from the treatment information.

11. The health care system of claim 8, wherein an artificial intelligence (AI) robot is provided in the selected treatment booth, and
the AI robot is configured to treat the user in the selected treatment booth according to the received treatment information.

12. The health care system of claim 8, wherein the treatment information includes information on a site of pain of the user, information on a treatment site of the user, and treatment method information.

13. The health care system of claim 8, wherein the health care server is configured to generate, if a reservation for the treatment is made, reservation information and transmit the generated reservation information to the user terminal.

14. The health care system of claim 8, wherein the health care server is configured to generate, if a reservation for the treatment is made, temporary visitor code information based on identification (ID) information of the selected treatment booth, reservation date and time information for the treatment, and ID information of the user, map the ID information of the selected treatment booth, the reservation date and time information for the treatment, and the ID information of the user, record the mapped information in a mapping table, transmit the generated temporary visitor code information to the user terminal, check, if an admission verification request and information obtained by the selected treatment booth by scanning a visitor code of the user terminal are received from the selected treatment booth, whether the obtained information is present in the mapping table and whether the time of the admission verification request matches the reservation date and time information, and verify an admission of the user based on a result of the checking.
